(19) United States
(12) Patent Application Publication
(10) Pub. No.: US 2017/0266007 A1
(43) Pub. Date: Sep. 21, 2017

(54) BONE SCREW

(71) Applicant: MATERIALISE N.V., Leuven (BE)

(72) Inventors: Frederik GELAUDE, Herent (BE); Tim CLIJMANS, Opvelp (Bierbeek) (BE)

(73) Assignee: MATERIALISE N.V., Leuven (BE)

(21) Appl. No.: 15/531,499

(22) PCT Filed: Nov. 27, 2014
(86) PCT No.: PCT/EP2014/075823

§ 371 Date: May 30, 2017

(51) Int. Cl. A61F 2/30; A61B 34/10; A61B 17/86

(52) U.S. Cl. CPC A61F 2/30749; A61B 17/8625; A61B 17/864; A61F 2/3094; A61B 17/866; A61B 17/863; A61B 34/10; A61F 2002/30011; A61F 2002/30004; A61F 2002/30948; A61F 2002/30952; A61F 2002/30985; A61F 2002/30962; A61F 2002/3097; A61F 2002/30968; A61B 2034/104; A61B 2034/108

(57) ABSTRACT

Method for manufacturing a bone pin for connection to a bone, particularly for fixing an implant to a bone, the bone pin having an implant contacting part arranged to contact the implant in connected situation and a bone contacting part arranged to engage the bone in connected situation, wherein the method comprises the steps of:—providing bone information which is indicative for the bone which the bone contacting part is arranged to engage;—providing a bone contacting part which is customized on the basis of the bone information for engaging said bone;—providing an implant contacting part; and—assembling the bone contacting part and the implant contacting part for manufacturing the pin.

The present invention relates to a method for manufacturing a bone pin for connecting an implant or similar device to a bone and a bone pin as such.

For connecting implants to the bone, for instance bone implants for repairing bone defects, bone screws are typically used. These bone screw typically have an outwardly extending head, with regard to the remainder of the screw, arranged to contact the implant in a hole in said implant. The part distally of this implant connecting part is usually arranged to engage the bone for connecting and fixating the implant to the bone. This distal part is typically provided with a sharp tip or point.

The success rate of for instance such a bone reconstructing operation using an implant is largely dependent on the manner of fixing the implant to the bone and whether the implant in implanted situation is capable of withstanding the loading conditions. For instance, loosening of the implant due to incorrect fixation of the implant by a bone screw is detrimental to the success rate of such a reconstructing operation. More specifically, screw fixation serves at short term fixation of the implant, whereas biologic fixation of the implant serves as mid to long term fixation. If an implant only relies on traditional screws for fixation, these screws will typically fail in mid term, and with this the entire reconstruction.

It is therefore a goal of the present invention, next to other goals, to provide an enhanced bone screw which at least partially alleviates the above problem.

This goal, amongst other goals, is met by a method for manufacturing a bone pin for connection to a bone, more particularly for fixing an implant to a bone, the bone pin having an implant contacting part arranged to contact the implant in connected situation and a bone contacting part arranged to engage the bone in connected situation, wherein the method comprises the steps of:
providing bone information which is indicative for the bone which the bone contacting part is arranged to engage;
providing a bone contacting part which is customized on the basis of the bone information for engaging said bone;
providing an implant contacting part; and
assembling the bone contacting part and the implant contacting part for manufacturing the pin.

According to the invention, at least the bone contacting part of the pin is customized for the intended application, i.e. for the bone which said bone contacting part is arranged to engage. Thereto, prior to assembling the pin, or otherwise manufacturing as will be explained in greater detail below, information about the bone, for instance in terms of quality, density or structure is obtained. This decreases the risk of implant failure due to insufficient fixation by the pin. According to a preferred embodiment, the bone contacting part is customized in terms of length, porosity, material, threading, diameter or core diameter, or a combination thereof.

The pin according to the invention may be provided with a single bone contacting part with essentially homogeneous properties. These properties are then determined on the basis of the bone the part is intended to engage. The properties of the bone may however vary along the bone contacting part in use. A single bone contacting part may hereto be provided with different properties, for instance changing gradually along the length in accordance with the changing properties of the bone. It is however also possible that the properties of the bone contacting part are determined as an optimum for engaging the bone having different properties.

It is however preferred if the pin according to the invention is arranged to improve fixation of the implant by locally adapting the parts of the pin in contact with the bone to the locally varying bone characteristics. Therefore, according to a preferred embodiment, the pin comprises at least two different bone contacting parts arranged for engaging at least two different parts of the bone in connected situation. This allows adaptation or customization of at least one of the bone contacting parts to the corresponding bone part. It is hereby preferred if the different bone contacting parts extend in the longitudinal direction of the pin with respect to each other.

Although it may be possible that the two bone parts have similar characteristics, i.e. have a substantially homogeneous structure, it may still be advantageous to provide different bone contacting parts having different functions. A first bone contacting part may for instance be arranged to promote bone ingrowth, and thereto has an open structure as will be discussed in greater detail below, while a second bone contacting part, which engages bone having similar properties, is arranged to enhance initial fixation and is thereto provided with a tapering diameter and/or threading. The different bone contacting parts may therefor be arranged to fulfill different functions and may therefore be accordingly customized thereto, such that this results in bone contacting parts having different properties.

However, in case the different parts of the bone have substantially different bone characteristics, it is preferred if the method further comprises providing bone information which is indicative for the bone characteristics of both parts of the bone. It is then preferred if the bone contacting parts are customized on the basis of the bone information for engaging said different bone parts having different characteristics. By adapting the bone contacting parts to the varying bone characteristics, the fixation of these bone contacting parts to these bone parts is improved, thereby improving the fixation of the pin as a whole.

Providing the bone information may comprise simply (intra-)operative measurements such as cortex thickness measurement using a gauge or impression pin or by pre-drilling. On the basis of this measurement, a suitable bone contacting part may be selected, for instance from a plurality of bone contacting parts at the disposal of the user. The pin may then be assembled, for instance by interlocking the implant contacting part with the bone contacting for manufacturing the pin.

However, according to preferred embodiment, the step of providing bone information comprises medical imaging for obtaining the bone characteristics of at least the bone surrounding the bone contacting parts of the pin. Using medical imaging, such as a CT- or MRI-scan or similar, provides detailed information about the bone into which the pin is to be inserted. It is in particular preferred if the bone information comprises bone (mineral) density data, for instance obtained by a DEXA- or CT-scan. Providing the bone information more preferably comprises providing a data file obtained earlier by medical imaging. The step of providing the bone contacting part imaging therefore does not need to take place directly following the step of imaging. More specifically, it is preferred if the pin is prefabricated for manufacturing a patient specific customized pin.

According to a further preferred embodiment, the step of providing the bone information comprises generating a three-dimensional bone model of at least the part of the bone into which the pin is to be inserted and is in contact with in the connected situation. Preferably, the step of providing the three-dimensional bone model comprises the step of obtaining an image of the bone into which the pin is to be inserted. Digital patient-specific image information can be provided by any suitable means known in the art, such as for example a computer tomography (CT) scanner, a magnetic resonance imaging (MRI) scanner, an ultrasound scanner, or a combination of Roentgenograms. A summary of medical imaging has been described in “Fundamentals of Medical imaging”, by P. Suetens, Cambridge University Press, 2002.

For example, the step of obtaining an image of the bone and the defect therein may comprise the steps of obtaining 2D datasets of the bone and reconstructing a 3D virtual bone model from said 2D datasets. Indeed, the first step in a planning is the construction of a 3D virtual model of the bone. This reconstruction starts with sending a patient to a radiologist for scanning, e.g. for a scan that generates medical volumetric data, such as a CT, MRI scan or the like. The output of the scan can be a stack of two-dimensional (2D) slices forming a 3D data set. The output of the scan can be digitally imported into a computer program and may be converted using algorithms known in the field of image processing technology to produce a 3D computer model of a relevant bone. Preferably, a virtual 3D model is constructed from the dataset using a computer program such as Mimics™ as supplied by Materialise N.V., Leuven, Belgium. Computer algorithm parameters are based on accuracy studies, as for instance described by Gelaude at al. (2008; Accuracy assessment of CT-based outer surface femur meshes Comput. Aided Surg. 13(4): 188-199). A more detailed description for making a perfected model is disclosed in U.S. Pat. No. 5,768,134 entitled ‘Method for making a perfected medical model on the basis of digital image information of a part of the body’. Once the three-dimensional model of the bone is reconstructed for instance as disclosed in Gelaude et al. (2007; Computer-aided planning of reconstructive surgery of the innominate bone: automated correction proposals Comput. Aided Surg. 12(5): 286-94) and is preferably combined with bone density data, the pin, in particular the bone contacting part(s) thereof can be provided, preferably by designing the bone parts taking the local bone information into account.

It is hereby preferred that also a model of the implant to be fixed to the bone is included in, or is associated with, the model. The design of the implant may already be available in a model, for instance in the case when the implant is a customized implant which is designed on the basis of the bone model. With this combined model of the bone and the implant to be fixed, it is possible to accurately determine the screw trajectories for a plurality of screws. Based upon this planned trajectories, the relevant bone information from the model, or perhaps any other source, may be used for designing the pin, in particular for designing the bone contacting parts thereof.

The planning of the screw trajectories to this end preferably includes the step of determining a length of a screw trajectory such that the screw extends through at least two different parts of the bone, preferably one part having a higher density such as cortical bone, and one part having a lower bone mineral density such as trabecular bone. This allows improved retention, as different bone contacting parts can be provided in accordance with the different types of bone. A further preferred embodiment of the method therefor further comprises the step of providing a pin having length determined on the basis of the bone information, wherein the pin is designed to have sufficient length such that the bone contacting parts thereof extend through at least two different types of bone.

To improve the fixation of a pin by adapting at least the bone contacting part thereof, the step of providing the bone contacting parts preferably comprises any of the following steps of:
a. providing a bone contacting part with threading in case the bone part to be engaged is cortical bone or other bone with a high bone mineral density;
b. providing a bone contacting part having a porous microstructure in case the bone part to be engaged is trabecular bone or other bone with a lower bone mineral density.
   More preferably, the method comprises both steps for the case the pin extends, or is to extend, through both types of bone. Providing a bone contacting part with threading for a part which is to contact cortical or other high density bone is found to enhance the fixation of the pin. This part may be connected to the bone by threading the pin into the bone, wherein the threaded part engages into the relatively stiff cortical bone to form a good initial connection therewith. A bone contacting part which is designed to contact trabecular bone on the other hand is designed to have a porous microstructure, for instance in form of a repeating microstructure, to promote bone ingrowth. This porous bone contacting part, of which at least the outer sections are arranged to contact the bone have a porous microstructure, therefor enhances the fixation over time, i.e. after sufficient ingrowth of bone into the porous structure.

A preferred embodiment of a pin according to the invention comprises a bone contacting part having a porous microstructure for bone ingrowth to enhance fixation, in particular over time. At least the outer part of such a bone contacting part thereto has an open structure. It is for instance possible that a more inner part, seen in radial direction along the longitudinal direction of the pin, is more or even completely solid for support. The pin, or at the porous bone contacting part, is thereto provided with a supporting structure, for instance a supporting core, to provide sufficient structural strength.

The properties of the porous microstructure and/or the supporting structure may be optimized based on predicted loading conditions of the implanted pin. A numerical model of the pin in connected state may for instance be created to calculate the loading conditions in use. Based on the loading conditions, the microstructure and/or the reinforcement structure may be adapted by adapting local material properties of the part, such as material type and/or Young's modulus, and/or by reinforcing the part by adapting the local density of the microstructure of the part. This method is disclosed in WO 2013/170872 in the name of the applicant and is hereby incorporated by reference.

It is however also possible to provide a bone contacting part having a porous structure which is also provided with threading. This allows a connection by screwing, while also allowing bone ingrowth. In particular in this embodiment, it is preferred to a have stiffening or supporting structure in at least the porous part to provide sufficient structural strength to allow screwing.

A further preferred embodiment of the method further comprises the step of providing a connecting part, wherein a connecting part of the pin is arranged to interact with another pin and is thereto provided with a receptacle for receiving another pin, wherein the step of assembling further comprises assembling the connecting part. The pin according to this embodiment is designed to interact with another pin, in particular another pin for fixing the implant. Connecting or having two screws interact in terms of contact, improves the fixation of the implant. In the pre-operative planning of the screw trajectories as mentioned above, in particular on the basis of the bone model, preferably in combination with the implant, at least two screw trajectories are planned to intersect. At least one of the screws for one of these trajectories is then designed to interact by at least partly receiving the other screw. The receptacle may for instance comprise threading or a similar connection, which is arranged to cooperate with for instance an end or another part of the other screw for interconnection of the two screws. The receptacle may further be arranged for creating a passage for the other screw. For instance, the receptacle may include a surface structure, for instance a groove or blind hole or a substantially smooth outer surface of a part provided with threading, such that the other screw does not interfere with the threading.

It is however also possible that the receptacle comprises a through hole for receiving another pin. The through hole in the connecting part is thereto designed to be sufficiently large to receive and pass through the other pin. The through hole may also be provided with threading or a similar connection to improve the interconnection between the two screws.

According to a further preferred embodiment, at least the step of providing the bone contacting part, preferably also the step of assembling or manufacturing the pin, comprises using a three-dimensional printing technique, also referred to as rapid manufacturing technique, layered manufacturing technique, additive manufacturing technique or material deposition manufacturing technique. Preferably the method hereto comprising designing the different parts of the pin, for instance the bone contacting part(s), the implant contacting part and possibly the connecting parts, to subsequently print the pin as a whole on the basis of the design. The step of assembling may therefore comprise three-dimensional printing.

Rapid manufacturing includes all techniques whereby an object is built layer by layer or point per point by adding or hardening material (also called free-form manufacturing). The best known techniques of this type are stereolithography and related techniques, whereby for example a basin with liquid synthetic material is selectively cured layer by layer by means of a computer-controlled electromagnetic beam; selective laser sintering, whereby powder particles are sintered by means of an electromagnetic beam or are welded together according to a specific pattern; fused deposition modelling, whereby a synthetic material is fused and is stacked according to a line pattern; laminated object manufacturing, whereby layers of adhesive-coated paper, plastic, or metal laminates are successively glued together and cut to shape with a knife or laser cutter; or electron beam melting, whereby metal powder is melted layer per layer with an electron beam in a high vacuum.

In particular embodiments, Rapid Prototyping and Manufacturing (RP&M) techniques, are used for manufacturing (parts of) the pin according to the invention. Rapid Prototyping and Manufacturing (RP&M) can be defined as a group of techniques used to quickly fabricate a physical model of an object typically using three-dimensional (3-D) computer aided design (CAD) data of the object. Currently, a multitude of Rapid Prototyping techniques is available, including stereo lithography (SLA), Selective Laser Sintering (SLS), Fused Deposition Modeling (FDM), foil-based techniques, etc. A common feature of these techniques is that objects are typically built layer by layer.

Stereo lithography (SLA), presently the most common RP&M technique, utilizes a vat of liquid photopolymer “resin” to build an object a layer at a time. On each layer, an electromagnetic ray, e.g. one or several laser beams which are computer-controlled, traces a specific pattern on the surface of the liquid resin that is defined by the two-dimensional cross-sections of the object to be formed. Exposure to the electromagnetic ray cures, or, solidifies the pattern traced on the resin and adheres it to the layer below. After a coat had been polymerized, the platform descends by a single layer thickness and a subsequent layer pattern is traced, adhering to the previous layer. A complete 3-D object is formed by this process.

Selective laser sintering (SLS) uses a high power laser or another focused heat source to sinter or weld small particles of plastic, metal, or ceramic powders into a mass representing the 3-dimensional object to be formed.

Fused deposition modeling (FDM) and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically RP&M techniques start from a digital representation of the 3-D object to be formed, in this case the design of (parts of) the pin. Generally, the digital representation is sliced into a series of cross-sectional layers which can be overlaid to form the object as a whole. The RP&M apparatus uses this data for building the object on a layer-by-layer basis. The cross-sectional data representing the layer data of the 3-D object may be generated using a computer system and computer aided design and manufacturing (CAD/CAM) software.

The pin of the invention may be manufactured in different materials. Typically, only materials that are biocompatible (e.g. USP class VI compatible) with the human body are taken into account. Preferably the implant is formed from a heat-tolerable material allowing it to tolerate high-temperature sterilization. In the case SLS is used as a RP&M technique, the pin may be fabricated from a polyamide such as PA 2200 as supplied by EOS, Munich, Germany or any other material known by those skilled in the art may also be used.

The invention further relates to a bone pin or screw for fixing an implant a bone, in particular manufactured with a method according to the invention, the bone pin having an implant contacting part arranged to contact the implant in connected situation and a bone contacting part arranged to engage the bone in connected situation, wherein the bone contacting part is customized for engaging said bone. Providing a bone screw or pin which is at least partly customized to the bone into which the screw is to be inserted, allows an enhanced fixation of an implant to the bone. As mentioned above, a bone pin according to a preferred embodiment comprises a plurality of bone contacting parts, each customized for engaging said bone, wherein at least two of the bone contacting parts have different properties in terms of length, material, threading, diameter or core diameter, or a combination thereof. Such a pin can be customized to varying bone characteristics along the length of said pin, thereby enhancing fixation.

A particularly enhanced fixation is obtained, in particular in trabecular bone, if according to a further preferred embodiment the bone contacting part is at least partly manufactured from a porous material. In order to also improve fixation already on insertion, it is further preferred that the bone pin comprises at least one bone contacting part provided with threading and at least one bone contacting part having a porous microstructure.

Although it is possible that the threading is provided on another bone contacting part, for instance for engaging bone having a higher density as mentioned above, while the porous part engages or contacts trabecular bone, it may further be possible that the porous bone contacting part is provided with threading. A bone pin according to this embodiment may for instance consist of a single bone contacting part, next to for instance a sharp tip provided at the distal end. To prevent damage when applying higher forces, for instance torque for screwing said part provided with threading, a further preferred embodiment further comprises a reinforcing structure for reinforcing at least the porous bone contacting part.

To increase the fixation using at least two intersecting pins as mentioned above, a further preferred embodiment of the bone pin according to the invention further comprises a connecting part, wherein a connecting part of the pin is arranged to interact with another pin and is thereto provided with a receptacle for receiving another pin. The receptacle may comprise a through hole for receiving another pin.

The invention further relates to a kit of parts for assembling a bone pin according to the invention, comprising an implant contacting part and a bone contacting part, wherein the implant contacting part and/or the bone contacting part is provided with connecting means for interconnecting the parts.

The present invention is further illustrated by the following Figures, which show a preferred embodiment of the device according to the invention, and are not intended to limit the scope of the invention in any way, wherein:

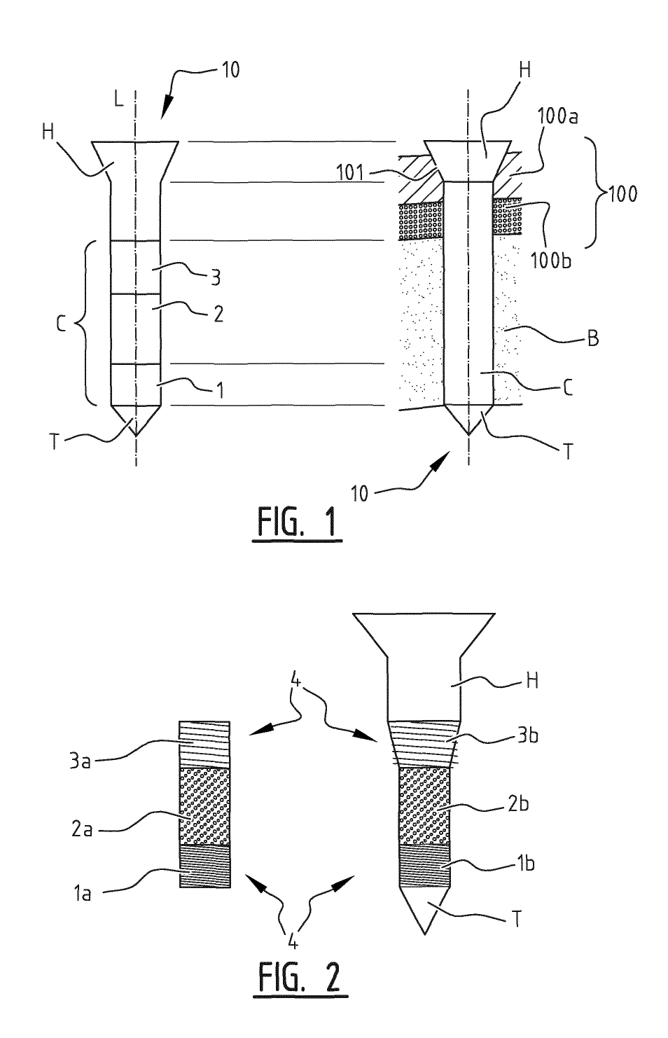

FIG. 1 schematically shows the different parts of the screw according to the invention;

FIG. 2 shows two embodiments of the invention;

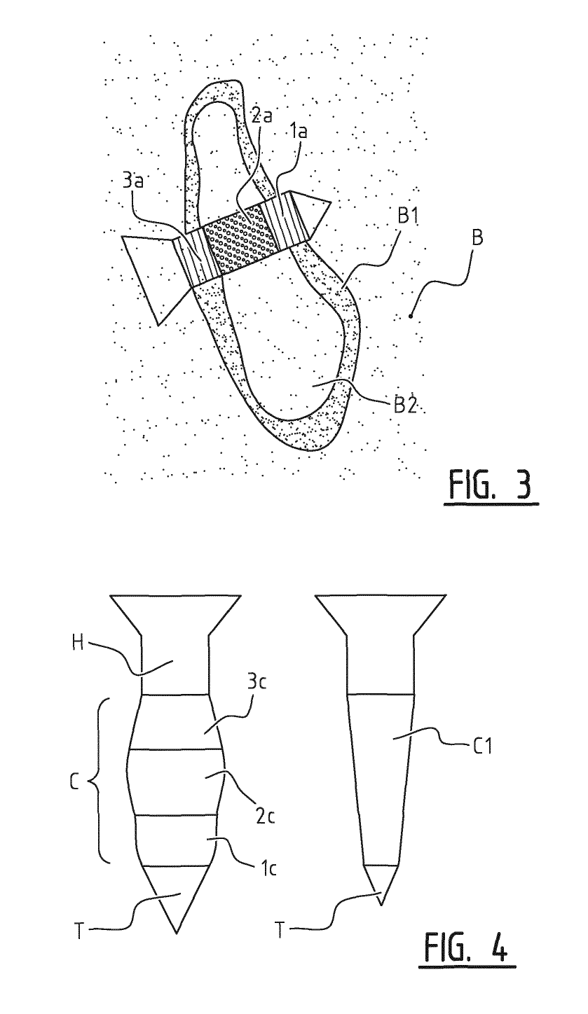

FIG. 3 shows the left screw of FIG. 2 in connected situation; and

FIGS. 4-9 schematically show further embodiments of the screw according to the invention.

In FIG. 1, the basic configuration of a bone screw 10 according to the invention is shown. The screw 10 is arranged to fixate an implant 100, see the right figure, to the bone B. The implant 100 in this example comprises a plate-like solid structure 100a and a porous structure 100b for bone ingrowth. The implant 100 is further provided with a tapering hole 101 for receiving an accordingly tapered head H of the screw 10.

The screw 10 is further provided with a sharp point T at the distal end, i.e. seen along the longitudinal direction L of the screw 10. In between the head H and the cylindrical part distally therefrom which are arranged to contact the implant 100 in connected state, hereafter referred to as the implant contact part of the screw 10, is provided a bone contacting part C, in this example subdivided in three part 1-3, although it will be apparent that the invention is not limited to a particular number of subdivisions of the bone contacting part C. Dividing the bone contacting part C in different sections or simply by providing a plurality of bone contacting parts 1-3 along the length L of the screw 10, allows customization of the screw 10 to varying bone characteristics the different parts are to engage in connected situation.

In the examples of FIG. 2 for instance, three bone contacting parts 1a, 2a, 3a and 1b, 2b and 3b are provided. The part 1a of the first example (on the left) is for instance provided with a thread 4 having a first pitch, while the third part 3a is provided with a thread 4 having a different, in this example larger, pitch. In between the parts 1a and 3a is provided a porous part 2a which is arranged to promote ingrowth in connected state. A similar screw is for instance shown in connected situation in FIG. 3. It is visible that the first and third parts 1a and 3a (in this example provided with a thread 4 having the same pitch) engage cortical bone B1, while the inner porous part 2a engage trabecular bone B2. The screw as shown in FIG. 3 is specifically designed to allow this engagement. More specifically, for the design of the screw, bone information, in this example bone mineral density data of the bone was available, such that the length of the screw and the division of the bone contacting parts 1a-3a could be chosen such that two threaded parts 1a and 3a engage cortical bone B1, while the porous part 2a engages spongy bone B2. The screw 10 is thus customized to the patient's bone into which the screw is designed to be inserted. The variant on the right of FIG. 2 varies in that the diameter of the third part 3b varies along the length L. Further examples of varying diameters of the parts 1c-3c are visible in FIG. 4. In the right of FIG. 4 it is further visible that also a single bone contacting part C1, in this example with decreasing diameter towards the tip T is possible.

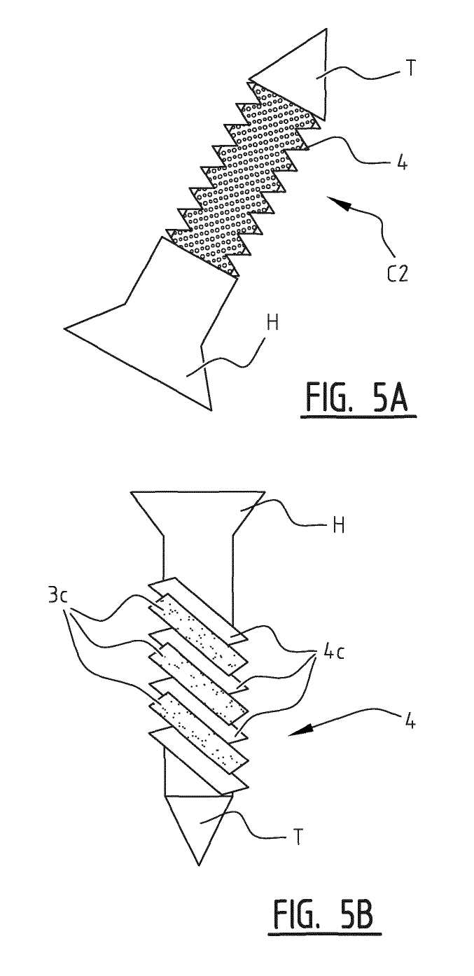

Also in the example of FIG. 5a the bone contacting part C2 is provided. Only in this example, this part is porous while this part is also provided with a thread 4. The tip T is preferably solid. Although it is not visible in this figure, the inner part of the bone contacting part C2 is provided with a solid structure to provide sufficient strength to the screw. FIG. 5b shows an example of bone contacting parts 3c, 4c which extend under an angle with respect to the longitudinal axis of the screw. The components 3c and 4c, of which the part 3c is porous and of which part 4c is solid, may for instance extend helically around the longitudinal axis such that the parts 4c form a threading 4.

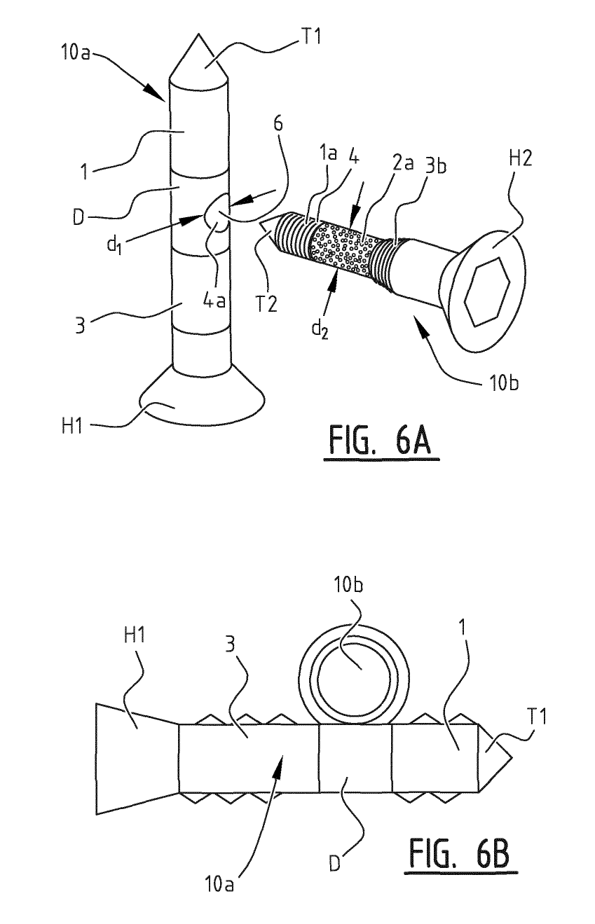

In the example of FIGS. 6a and 6b, a set of screws is shown which are arranged to cooperate and engage in connected situation. The first screw 10a in FIG. 6a is hereto, next to two not further specified bone contacting parts 1 and 3, provided with a part D which is arranged to connect to another screw 10b. The connecting part D is hereto provided with a through hole 6, which diameter d1 corresponds to a diameter d2 of the part 1a with which the connecting part D is arranged to engage. More specifically, during a pre-operative planning of the connection of an implant, the screws as shown in FIG. 6a are planned to intersect such that a part 1a extends through the hole 6. The hole 6 and/or the part 1a may thereto be provided with threading 4, 4a to enhance the fixation. As an alternative as shown in FIG. 6B, the screw contacting part D may be provided with a (locally) smooth surface, such that the second screw 10b can pass without substantial interference.

The screw according to the invention is preferably manufactured as a whole using a three-dimensional printing technique. The screw is thereto preferably first designed, in particular on the basis of image data as shown in FIG. 3, and subsequently printed. In particular the porous structures are very suitable to be printing in this way.

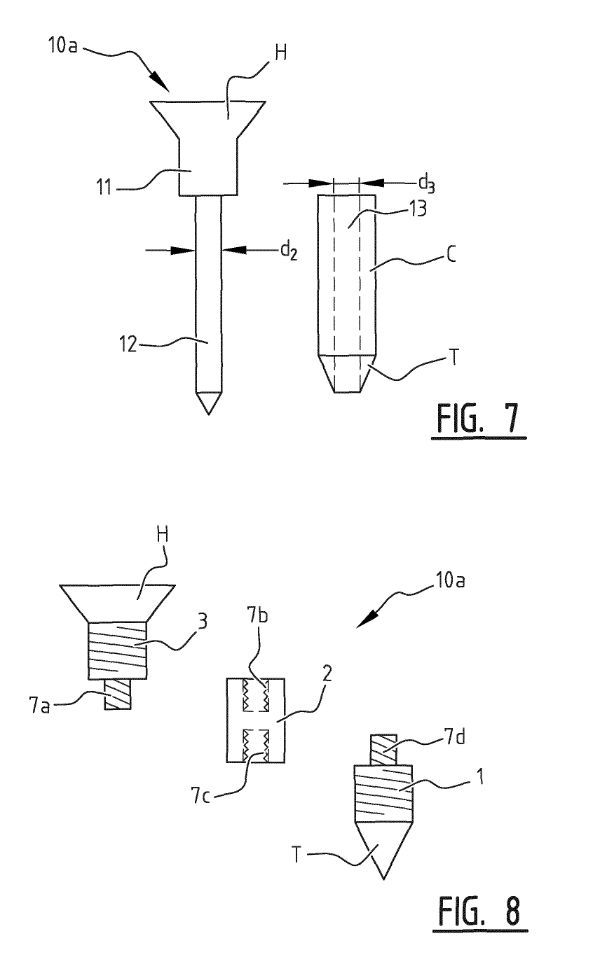

With reference to FIG. 7, it is however also possible that the different parts, for instance the bone contacting part C and main part 11, for instance provided with the head H and the most distal part of the tip and interconnected with a core 12, are provided separately. During an operation, the user may select a suitable bone contacting part, or a plurality thereof to combine, and assemble the screw 10a by inserting the core 12 into the correspondingly shaped hole 13, in terms of diameters d2 and d3.

An alternative is shown in FIG. 8, wherein different parts of the screw 10a can be assembled by threading 7a, 7b, 7c and 7d, the threading of which is in the same direction of the treads of the bone contacting parts 1 and 3 to prevent loosening of the parts upon screwing of the screw into the bone. Alternative connections may be used, such as a bayonet fitting.

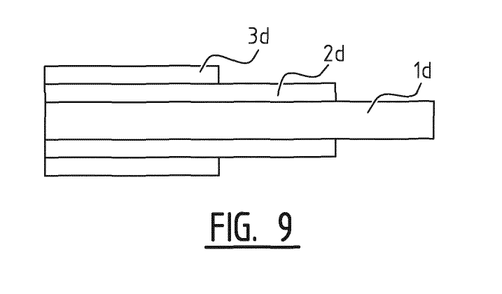

In FIG. 9, an embodiment of a pin is shown wherein the different bone contacting parts 1-3d extend radially with respect to each other and the longitudinal axis of the pin. This pin can either be screwed or inserted in the bone. The parts are hereto cylindrically shaped and manufactured to closely fit each other in the radial direction. Each of the parts 1-3d may have different properties adapted to the bone the parts are arranged to contact and/or the function the parts 1-3d have. The lengths are adjusted accordingly. It is for instance possible that first a solid part 1d, for instance provided with a tip (not shown) is inserted and that subsequently the second part 2d is advanced over the first part 1d. This part may for instance be a porous part for engaging porous bone. The last part 3d may for instance again be a solid part for engagement with an implant.

It is also possible that an outer part 3d has a length which is greater than an inner part. This outer part 3d then preferably extends beyond said inner parts, such that a hollow core at the tip is formed which can be filled with bone by bone ingrowth. A similar connection with improved bone-ingrowth can be obtained if after insertion of for instance the combination of FIG. 9, an inner part 1d is withdrawn after insertion. This part then may have a length similar to for instance the more outer part 2d. This hollow core may then be filled with bone, which improves the connection of the pin over time.

The present invention is not limited to the embodiment shown, but extends also to other embodiments falling within the scope of the appended claims.

1. Method for manufacturing a bone pin for connection to a bone, particularly for fixing an implant to a bone, the bone pin having an implant contacting part arranged to contact the implant in connected situation and a bone contacting part arranged to engage the bone in connected situation, wherein the method comprises the steps of:
providing bone information which is indicative for the bone which the bone contacting part is arranged to engage;
providing a bone contacting part which is customized on the basis of the bone information for engaging said bone;
providing an implant contacting part; and
assembling the bone contacting part and the implant contacting part for manufacturing the pin.

2. Method according to claim 1, wherein the bone contacting part is customized in terms of length, porosity, material, threading, diameter or core diameter, or a combination thereof.

3. Method according to claim 1 or 2, wherein the pin comprises at least two different bone contacting parts arranged for engaging at least two different parts of the bone in connected situation, the different parts of the bone having different bone characteristics, wherein the method further comprises providing bone information which is indicative for the bone characteristics of both parts of the bone, further comprising providing bone contacting parts which are customized on the basis of the bone information for engaging said different bone parts.

4. Method according to claim 3, wherein the different bone contacting parts extend in the longitudinal direction of the pin with respect to each other.

5. Method according to any of the preceding claims, wherein the step of providing bone information comprises (intra-)operative measurements

6. Method according to any of the preceding claims, wherein the step of providing bone information comprises medical imaging for obtaining the bone characteristics of at least the bone surrounding the bone contacting parts of the pin.

7. Method according to claim 6, wherein the bone information comprises bone density data.

8. Method according to claim 6 or 7, further comprising the step of providing a pin having length determined on the basis of the bone information, wherein the pin is designed to have sufficient length such that the bone contacting parts thereof extend through at least two different types of bone.

9. Method according to any of the preceding claims, wherein the step of providing the bone contacting parts comprises any of the following steps of:
a. providing a bone contacting part with threading in case the bone part to be engaged is cortical bone or other bone with a high bone mineral density;
b. providing a bone contacting part having a porous microstructure in case the bone part to be engaged is trabecular bone or other bone with a lower bone mineral density.

10. Method according to at least claims 3, 8 and 9, wherein the pin comprises at least one bone contacting part provided with threading and at least one bone contacting part having a porous microstructure.

11. Method according to any of the preceding claims, further comprising the step of providing a connecting part, wherein a connecting part of the pin is arranged to interact with another pin and is thereto provided with a receptacle for receiving another pin, wherein the step of assembling further comprises assembling the connecting part.

12. Method according to claim 11, wherein the receptacle comprises a through hole for receiving another pin.

13. Method according to any of the preceding claims, wherein at least the step of providing the bone contacting part, preferably also the step of assembling or manufacturing the pin, comprises using a three-dimensional printing technique.

14. Bone pin for fixing an implant to a bone, the bone pin having an implant contacting part arranged to contact the implant in connected situation and a bone contacting part arranged to engage the bone in connected situation, wherein the bone contacting part is customized for engaging said bone.

15. Bone pin according to claim 14, comprising a plurality of bone contacting parts, each customized for engaging said bone, wherein at least two of the bone contacting parts have different properties in terms of length, porosity, material, threading, diameter or core diameter, or a combination thereof.

16. Bone pin according to claim 14 or 15, wherein the bone contacting part is at least partly manufactured from a porous material.

17. Bone pin according to claim 16, wherein the bone pin comprises at least one bone contacting part provided with threading and at least one bone contacting part having a porous microstructure.

18. Bone pin according to claim 16, wherein the porous bone contacting part is provided with threading.

19. Bone pin according to claim 18, further comprising a reinforcing structure for reinforcing at least the porous bone contacting part.

20. Bone pin according to any of the preceding claims, further comprising a connecting part, wherein a connecting part of the pin is arranged to interact with another pin and is thereto provided with a receptacle for receiving another pin.

21. Bone pin according to claim 20, wherein the receptacle comprises a through hole for receiving another pin.

22. Bone pin according to any of the preceding claims, wherein at least the bone contacting part, preferably whole the pin, is manufactured using a three-dimensional printing technique.

23. Kit of parts for assembling a bone pin according to any of the preceding claims, comprising an implant contacting part and a bone contacting part, wherein the implant contacting part and/or the bone contacting part is provided with connecting means for interconnecting the parts.
